(19) United States
(12) Patent Application Publication
(10) Pub. No.: US 2022/0074919 A1
(43) Pub. Date: Mar. 10, 2022

(54) Toilet Wastewater Collection and Testing

(71) Applicant: Hall Labs, LLC, Provo, UT (US)

(72) Inventors: David R. Hall, Provo, UT (US); K. Jeffrey Campbell, Spanish Fork, UT (US)

(21) Appl. No.: 17/468,639

(22) Filed: Sep. 7, 2021

Related U.S. Application Data
(60) Provisional application No. 63/074,608, filed on Sep. 4, 2020.

(51) Int. Cl. G01N 33/483; G01N 33/493; C12Q 1/04; C12Q 1/70

(52) U.S. Cl. CPC G01N 33/4833; G01N 33/493; E03D 9/00; C12Q 1/70; C12Q 1/04

(57) ABSTRACT

A method for collecting and testing an excreta sample from a user is disclosed. The system comprises a bowl having a volume of water for receiving excreta from a user, the excreta and water forming a volume of wastewater; a first sensor for detecting the addition of excreta to the water in the bowl; an orifice for collecting a sample of wastewater; wherein the sample is analyzed to determine a property of the excreta, which is used to obtain health and wellness information about the user. The method comprises providing a toilet comprising a bowl having a volume of water for receiving excreta from a user, the excreta and water forming a volume of wastewater; a first sensor for detecting the addition of excreta to the water in the bowl; an orifice for collecting a sample of wastewater; receiving excreta into the bowl; creation of wastewater through water contacting the excreta and excreta matter dispersing into the water; drawing a portion of the wastewater into the orifice to collect a sample of the wastewater; and testing the sample to gather health and wellness information about the user derived from the excreta matter in the wastewater.

### CROSS-REFERENCE TO RELATED APPLICATIONS

This application claims the benefit under 35 U.S.C. 119(e) of U.S. Provisional Patent Application No. 63/074,608 titled “Toilet Wastewater Collection and Testing” filed on 4 Sep. 2020, which disclosure is incorporated herein by reference in its entirety.

### TECHNICAL FIELD

The present disclosure relates to analytical toilets. More particularly, it relates to analytical toilets equipped to provide health and wellness information to the user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Just a few examples of smart toilets and other bathroom devices can be seen in the following U.S. patents and Published applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; and US 2018/0165417, entitled “Bathroom Telemedicine Station.” The disclosures of all these patents and applications are incorporated by reference in their entireties.

### SUMMARY

In a first aspect, the disclosure provides a method of collecting and testing an excreta sample from a user. The method includes providing a toilet including a bowl for receiving excreta from a user; a standing water level in the bowl; and an orifice below the standing water level and in fluid communication with the location of the standing water. It further includes receiving excreta into the bowl; collecting excreta received by the bowl into the standing water; allowing the excreta to interact with the standing water to create a wastewater mixture comprising excreta dispersed in the standing water; drawing a portion of the wastewater mixture into the orifice to collect a sample of the wastewater mixture; and testing the sample to gather health and wellness information about the user derived from the excreta in the mixture.

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is an isometric view of one exemplary embodiment of a toilet.

FIG. 2 is a top view of the toilet of FIG. 1.

FIG. 3 is a view of the bottom of the seat and lid of the toilet of FIG. 1.

FIG. 4 is a view from the side of the toilet of FIG. 1 with the cover removed.

FIG. 5 is an isometric view of a second exemplary embodiment of a toilet.

FIG. 6 is a top view of the toilet of FIG. 7.

FIG. 7 is a view of the bottom of the seat of the toilet of FIG. 7.

FIG. 8 is a partial view of the toilet of FIG. 7 with the cover removed.

FIG. 9 is a detail view of one exemplary embodiment of a handle.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” or “frame” refers to the portion of the toilet below and around the bowl supporting it.

As used herein, the term “user” refers to any individual who interacts with the toilet and deposits excreta therein.

As used herein, the term “excreta” refers to any substance released from the body of a user including urine, feces, menstrual discharge, saliva, expectorate, and anything contained or excreted therewith.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “imaging sensor” is meant to refer to any device for detecting and/or measuring a property of a person or of a substance that relies on electromagnetic radiation of any wavelength (e.g., visible light, infrared light, x-ray) or sound waves (e.g., ultrasound) to view the surface or interior of a user or substance. The term “imaging sensor” does not require that an image or picture is created or stored even if the sensor is capable of creating an image.

As used herein, the term “data connection” and similar terms are meant to refer to any wired or wireless means of transmitting analog or digital data and a data connection may refer to a connection within a toilet system or with devices outside the toilet.

As used herein, the terms “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, the term “analyte” is meant to refer to a substance whose chemical constituents are being identified and measured.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, the term “microfluidics” is meant to refer to the manipulation of fluids that are contained to small scale, typically sub-millimeter channels. The prefix “micro” used with this term and others in describing this invention is not intended to set a maximum or a minimum size for the channels or volumes.

As used herein, the prefix “nano” is meant to refer to something in size such that units are often converted to the nano-scale for ease before a value is provided. For example, the dimensions of a molecule may be given in nanometers rather than in meters.

As used herein, “bind” and similar variants are meant to refer to the property of facilitating molecular interaction with a molecule, such as interaction with a molecular biomarker.

As used herein, “wastewater” is meant to refer to a fluid mixture collected in a bottom portion of the bowl, which may include standing water in the bowl and excreta collected therein. The mixture may be homogeneous (hereafter referred to as a “solution”) or it may be non-homogeneous.

### EXEMPLARY EMBODIMENTS

The present disclosure relates to a toilet which is capable of collecting a sample of wastewater so the sample can be tested to gain health and wellness information about a user from their excreta.

An analytical toilet can collect a sample of urine before the urine reaches the standing water at the bottom of the bowl. It can be much more difficult and/or undesirable to collect a sample of feces before the feces reaches the standing water in the bowl. A device used to collect a sample of feces before it reaches the standing water may have many drawbacks, including being difficult to implement, harder to clean, and less sanitary as well as increase a user's exposure to unpleasant odors from the feces. Additionally, preference may be given to an implementation that prevents or limits the amount of toilet paper and other foreign matter in the sample.

Various things can happen when excreta mixes with standing water. For excreta that is liquid-like, such as urine, sweat, and blood, the excreta likely to disperse into the water and may even form a homogeneous solution with the water. More gel-like or solid-like excreta, such as feces, spit, and menstrual discharge, is likely to retain some of the excreta in a cohesive shape or structure in the water, but portions like small particulates may disperse into the water. In some cases, these particulates may contain relevant biomarkers, analytes, or other matter that can be tested to gain health and wellness information about the user, including whole or partial pathogens. Preferably, the excreta is allowed to mix into the standing water without additional mixing, force, or other energy being applied to the excreta. Alternatively, additional mixing, force, or other energy may be added to help break up the excreta and or disperse it into the standing water. For example, a jet or other device may physically contact excreta to break it up. High temperature may be used to change the state of the excreta (such as liquifying fats in the excreta). A mixer may help disperse excreta elements more evenly in the standing water, such as a low-pressure waterjet or an agitator.

A toilet which collects a sample of wastewater may have a mechanism for collecting the sample. This mechanism may draw off a stream of the sample during a period of time or may collect a specific volume. Such a mechanism may be an orifice that is exposed to the wastewater. Such an orifice may permanently be positioned in the bowl below the standing waterline or may be moved into position in preparation for taking a sample. The mechanism may include a pumping action, such as capillary action or the creation of a lower pressure to draw the sample from the wastewater. The mechanism may also capture a specific volume such as with a scoop or cup-shaped structure moving relative to the wastewater.

A toilet with a permanent or fixed orifice may be preferred to reduce moving parts in the bowl. For example, the orifice may be built into or attached to the structure of the bowl. The orifice may be similarly integrated into another structure of the toilet that contains or catches the wastewater in the fluid pathway downstream from the bowl. Alternatively, the orifice may be part of a structure that moves relative to the wastewater. For example, the orifice may be on an arm or other structure that extends into the wastewater.

The orifice may be a nozzle formed in the bowl or downstream structure. The orifice may be near where the excreta is collected into the wastewater; one benefit of this is that an unmixed excreta/wastewater solution will be at a higher excreta concentration. The orifice may be far away from where the excreta is collected into the wastewater. One advantage of this is that it is also farther away from where toilet paper and other non-excreta could be collected and would therefore be less likely to be contaminated by the non-excreta. The orifice may also be a nozzle that screws into or otherwise attaches to the same. The orifice may be part of a port dedicated to wastewater collection or may share another function of the toilet, such as a cleaning port.

In one preferred embodiment, the toilet takes the sample of wastewater after excreta is deposited, but before the user adds potential contaminates, such as toilet paper. This collection could be activated manually by the user. This collection could also be done automatically by the toilet when the toilet determines that an excreta event has occurred. In one embodiment, this collection occurs at the initial wastewater/excreta location. Alternatively, the excreta may be moved from the initial location it settled in. One benefit of doing this is the user can add potential contaminants, such as toilet paper or medicaments, without contaminating the wastewater sample. Alternatively, the sample is collected after other contaminates, such as toilet paper, have been added to the wastewater.

Once the sample has been collected, a relevant analysis may test the sample. Furthermore, a sensor may capture results from the test. Any known excreta test may be performed in or by the toilet system. Preferably, the test will be one which directly analyzes the mixture. Alternatively, the test may require further processing to prepare the sample for analysis.

In one preferred embodiment, a controller receives data from sensors in the toilet, uses the data to determine various properties of the user based on their excreta, and/or uses the data to perform various actions. The controller may use sensor data to detect the presence of a user, detect and/or measure an excreta event, instruct the system to collect and/or analyze a sample of wastewater (i.e., excreta and toilet water), and measure user characteristics. The controller may process data locally and/or transmit it for analysis elsewhere and send information to devices or systems utilized by the user, their caretakers, or healthcare providers.

In one preferred embodiment, the sample is collected and the remaining wastewater is discarded from the toilet. Alternatively, additional samples are collected from the wastewater. In another alternative, the wastewater is moved to a secondary location for additional processing, collection, and/or testing.

The following US patent and Provisional Patent applications discuss various implementations of sensors which may be able to detect and/or measure properties of a user in various embodiments of toilets which could be modified to collect a sample of wastewater: U.S. patent application Ser. No. 16/818,900 titled “Toilet with Vascular Health Reporting” filed 13 Mar. 2020; Provisional Application No. 62/979,803 titled “Analytical Toilet for Assessing Analytes in Excreta” filed 21 Feb. 2020; Provisional Application No. 62/993,648 titled “Analytical Toilet for Detecting Viruses in Feces” filed 23 Mar. 2020; and Provisional Application No. 63/002,200 titled “Analytical Toilet for Detecting Viruses in Urine” filed 30 Mar. 2020. Each of these applications are incorporated into the specification herein by reference in their entirety.

In one preferred embodiment, the toilet is essentially any toilet that has a standing water level which collects excreta after the excreta has been received by the bowl and has a fluid connection for flushing or removing waste from the bowl but modified to collect a sample of the wastewater before flushing/removing the waste from the bowl.

In another preferred embodiment, the toilet is a urinalysis toilet that collects and/or performs analysis on urine before it has a chance to reach the standing water in the bowl. The urinalysis toilet would similarly be modified to collect a sample of wastewater once excreta has been collected in the standing water.

In yet another preferred embodiment, the toilet is designed to separate urine from feces. In such an embodiment, urine is less likely to be collected in standing water than feces. Regardless, the orifice may collect from the urine collection area. Alternatively, the orifice collects from the feces collection area.

In one embodiment, the sample of wastewater is filtered. One benefit of this is to specify the size of particles if a specific size range is desired. For example, when testing for pathogens, one may want to filter out particles larger than the pathogens being tested for. According to Wikipedia (https://en.wikipedia.org/wiki/List_of_pathogens_by_size), pathogens range in size from 17 nm for a porcine circovirus up to 25 m for a tapeworm (i.e., flatworm). Of the pathogens on the list, viruses range from 17 nm-120 nm; bacteria are 600 nm-10 μm; protists are 1 μm-14 μm; fungi are 10 μm-12 μm; metamonads are 10 μm-20 μm; and the non-flatworm animals are 0.3 mm-70 mm. Another potential range of sizes is that of cellular size. A human cell is approximately 5 μm-150 μm.

Additionally, it may be preferred to focus on pathogenic or cellular fragments and the filter may be sized smaller to accommodate the desired fragment size. For example, the size of a particular molecule may be used to determine filter size. The smallest molecule is the diatomic hydrogen (H2), with a bond length of 0.74 Å. Molecules commonly used as building blocks for organic synthesis have a dimension of a few Å to several dozen Å, or around one billionth of a meter. Molecules of many polymers can reach macroscopic sizes, including biopolymers such as DNA. The “Table of permselectivity for different substances” (https://en.wikipedia.org/wiki/Table_of_permselectivity_for_different_substances) lists many relevant substances that the kidneys may filter and their sizes. The list includes sodium (0.1 nm), potassium (0.14 nm), chloride (0.18 nm), urea (0.15 nm), glucose (0.33 nm), hemoglobin (3.25 nm), and serum albumin (3.55 nm).

Filters can be used to filter out larger particles. Filters can also be used to separate smaller particles from larger particles. A filter may also target a specific structure to filter, such as a filter that binds with a structure on a pathogen; a similar effect can be achieved on a macro scale with larger structures. Magnetism may also be used to filter. Additionally, filtration may include lasers and other powered devices. In one preferred embodiment, water or another fluid is pushed through the filter backward; one benefit of this cleaning the filter, especially by dislodging excreta particles, toilet paper, or other waste from the filter.

In one preferred embodiment, a cleaner may be used to remove waste from and/or sterilize portions of the toilet that contact the wastewater. The cleaner may be a consumable implemented as described in U.S. patent application Ser. No. 16/818,178 titled “Toilet with Sensor for Measuring Cleaning Consumable” filed 13 Mar. 2020, which is included herein by reference in its entirety. One benefit of this is to help prevent the growth of biomatter that may otherwise increase in the toilet. In one example, the water may run past a chemical tablet which can kill biomatter and pick up a small portion of the table on its way to sit in the bowl. A small amount of the chemical may prevent long term growth, but may be dilute enough not to compromise any samples or analysis being performed.

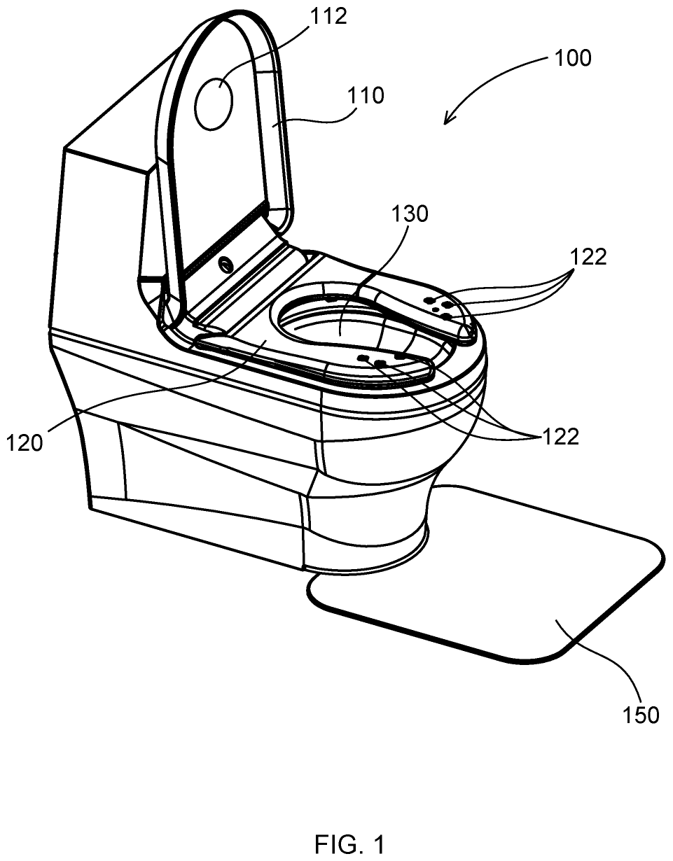

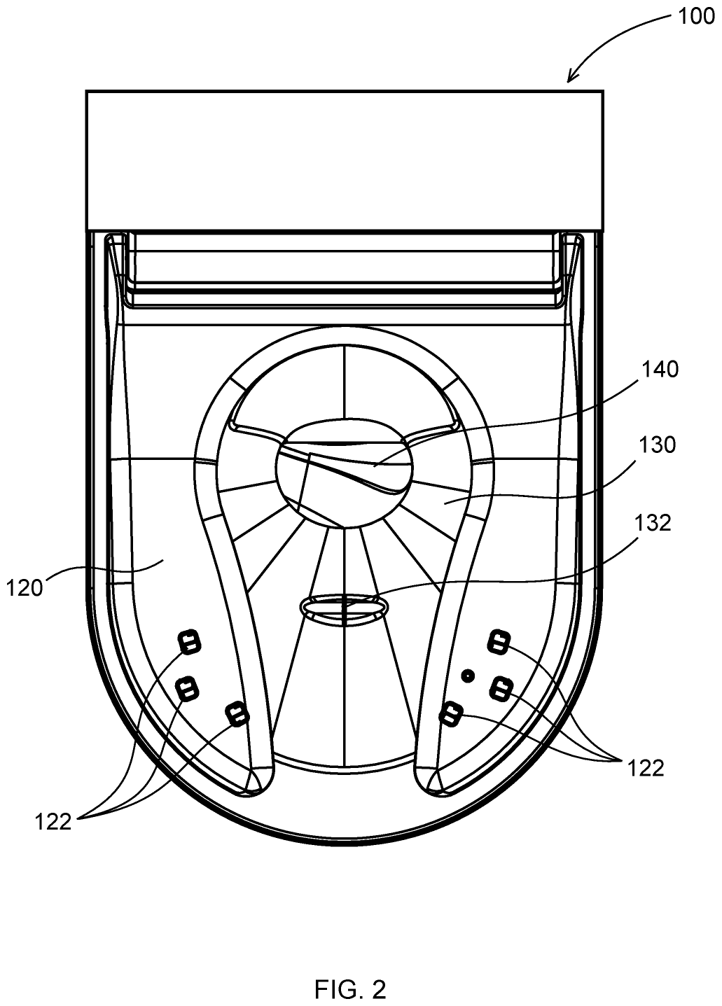

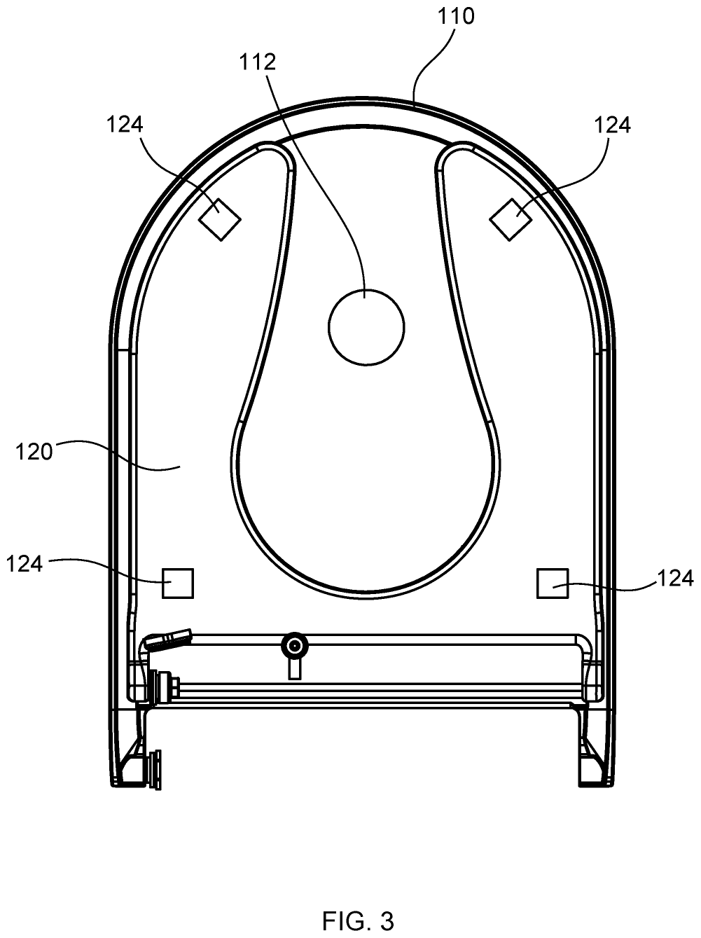

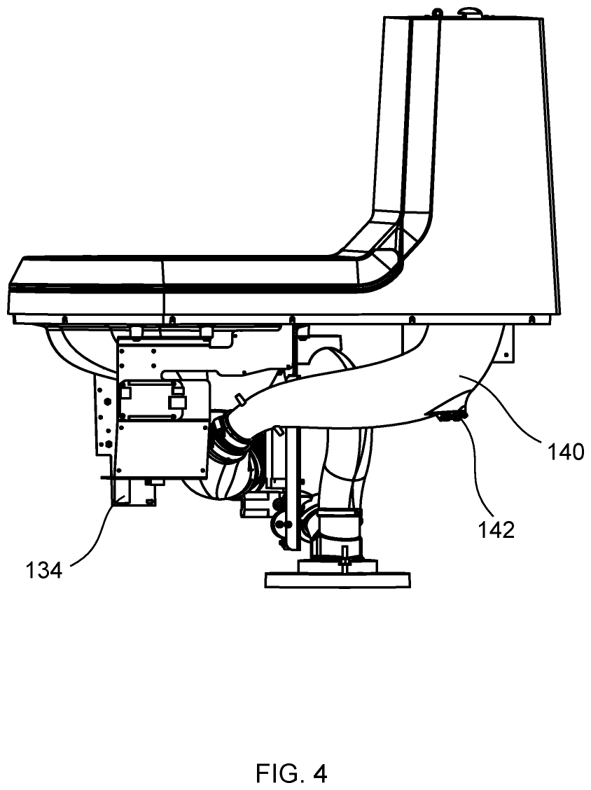

Now referring to FIGS. 1-4, one preferred embodiment of the toilet used in the system is shown. FIG. 1 shows an isometric view of toilet 100 with lid 110 open, showing seat 120 with multiple PPG sensors 122, bowl 130, and foot scale 150. Foot scale 150 may have a variety of sensors, such as sensors to determine a user's weight, image sensors, and electrical contacts. FIG. 2 shows a top view of toilet 100 with lid 110 open, showing seat 120 with multiple PPG sensors 122, bowl 130, and excreta volume measure tube 140. Bowl 130 includes urine slit 132, which captures urine for readings by spectrometer 134 and may also capture a sample of urine and remove the sample from the bowl to another location. FIG. 3 is a detail view of the underside of seat 120 with lid 110 behind seat 120. On the underside of seat 120 are weight sensors 124. Shown on lid 110 is acoustic sensor 112, which includes a microphone for recording audio sounds from a user's trunk portion of the body; acoustic sensor 112 could be supplemented with another type of sensor. FIG. 4 is a detail view showing some of the internal components of toilet 100, including excreta volume measure tube 140, excreta tube volume sensor 142, and spectrometer 134. The toilet may also have other sensors to detect properties of a user, including from a user's excreta. The toilet may also have a way of sampling and testing the wastewater in the bottom of bowl 130 and excreta measure tube 140 before and/or after it the water has interacted with excreta.

In one preferred embodiment, a user walks onto scale 150, and sits down on seat 120, leaving their feet on scale 150. While the user is using the toilet, PPG sensors 122 monitor the user's upper legs; weight sensors 124 monitor the portion of the user's weight on seat 120, including minor, apparent fluctuations that are a result of a user's cardiovascular activity; weight sensors 154 monitor the portion of the user's on foot scale 150, and bioimpedance sensors 152 determine the user's bioimpedance. After excreta has been deposited, the toilet samples the wastewater and either stores it for later testing or performs a test of the sample.

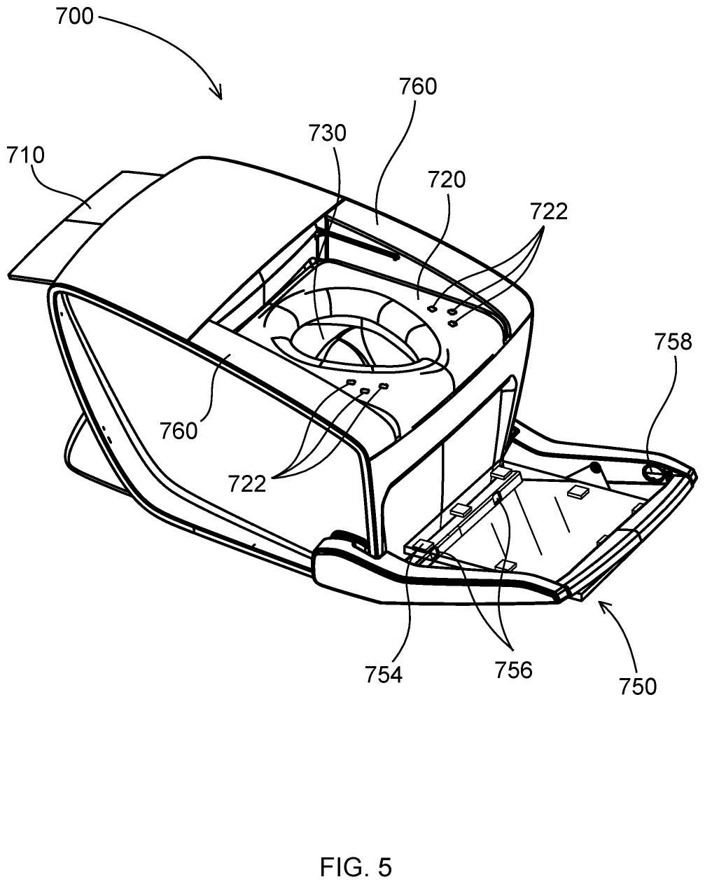

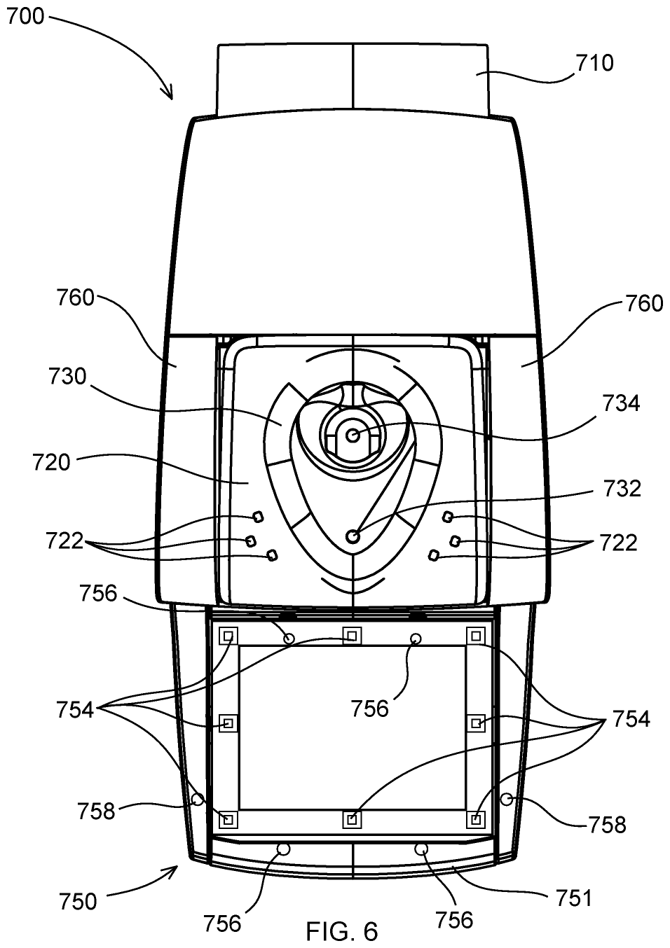

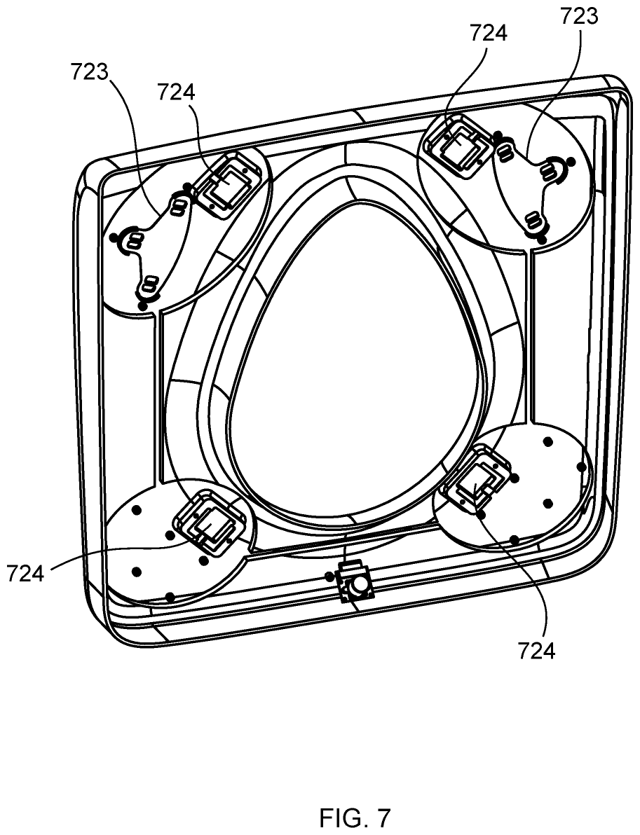

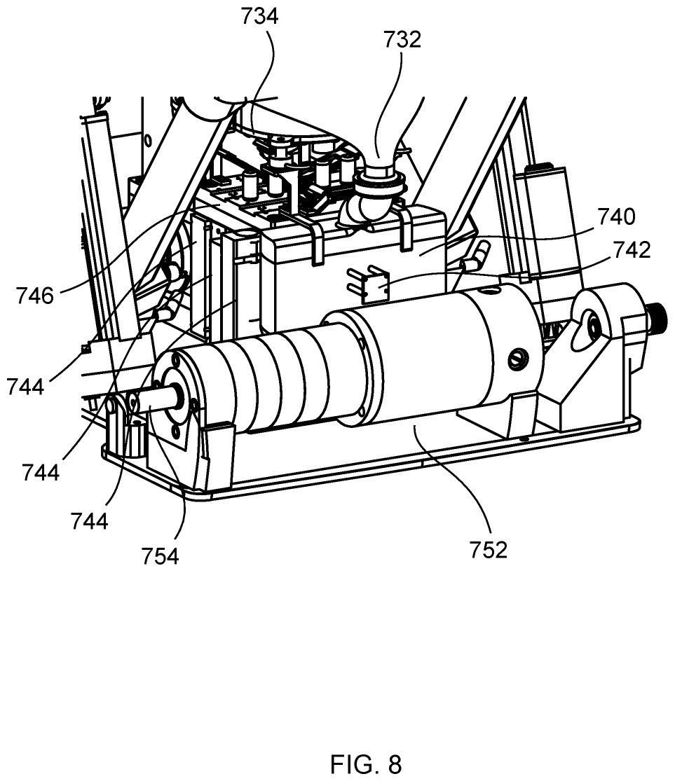

FIGS. 5-8 show another embodiment of the toilet. FIG. 5 shows an isometric view of toilet 700 with lid 710 open, showing seat 720 with multiple PPG sensors 722, bowl 730, foot platform 750, and handles 760. FIG. 6 shows a top view of toilet 700 with lid 710 open, showing seat 720 with multiple PPG sensors 722, bowl 730, foot platform 750, and handles 760. Bowl 730 includes urine receptacle 732 and fecal depository 734. In one preferred embodiment, handles 760 are in a recessed position and can be raised up relative to the toilet. FIG. 7 is a detail view of the underside of seat 720 showing weight sensors 724. FIG. 8 is a detail view showing some of the internal components of toilet 700, including urine receptacle 732, fecal depository 734, urine chamber 740, urine spectrometer 742, science centers 744, fluid chip receptacle 746, foot platform motor and sensor 752, foot platform motor shaft 753. Foot platform 750 includes frame 751, a glass plate resting on multiple weight sensors 754, foot IR sensors 756, and foot near-IR sensors 758. Additionally, visible light sensors may detect the feet too. In one preferred embodiment, science centers 744 and fluid chip receptacle 746 are used as part of excreta analysis in toilet 700, including urine samples and emulsified or otherwise processed excreta.

In one preferred embodiment, a user walks onto platform 750, sits down on seat 720, and platform 750 raises up so the user's feet easily stay on the glass plate. While the user is using the toilet, PPG sensors 722 monitor the user's upper legs; weight sensors 724 monitor the portion of the user's weight on seat 720, including minor, apparent fluctuations that are a result of a user's cardiovascular activity or other body tremors; weight sensors 754 monitor the portion of the user's on foot platform 750, and sensors 756 and 758 monitor the user's feet and lower legs. After excreta is deposited into the toilet, urine flows through urine receptacle 732 into urine chamber 740 where the urine can be sampled and/or tested; there may or may not be wastewater in urine chamber 740 with the urine. Feces is received by fecal depository 734 where it can be sampled and/or tested; there may or may not be wastewater on fecal depository 734 with the feces. At some point after, flush water can act to carry feces off fecal depository 734 to a secondary processing area where the feces and/or waste water can be sampled and/or tested; the water for the wastewater may solely be from the flush water or there may have been water already in the secondary processing area. Additionally, water jets and/or other process can be used to comminute the feces on the fecal depository or in the secondary processing area. Alternatively, the flush water and feces may go directly from the depository out of the toilet. There may also be toilet paper and other non-fecal/non-urine waste with the urine and/or feces.

In one preferred embodiment, sensors 756 and 758 are able to detect properties of the foot, including foot size and shape, coloring, and subdermal vascular properties. These sensors could be image sensors that capture visible light, IR light, or another part of the electromagnetic spectrum. They may also emit visible light, IR light, or another part of the electromagnetic spectrum. Images created from these sensors can undergo image recognition analysis, the results of which can be compared to preexisting data on the same to generate a report on a user's health. Preferably, the report includes information relative to a user's vascular health. Preferably, the comparison is performed by a neural net which has been trained to recognize commonalities to and differences from preexisting images. When the preexisting images are coupled with known health states and/or conditions of the person from whom the images came, the neural net can suggest correlations between the user's images and health states and/or conditions (including neutral or positive ones). Additionally, when the neural net has examined previous data from the same user, the neural net can compare the user's prior state to his or her current state to report on the relative change. Therefore, it may be useful for user data to be averaged, have the mean taken, used in creating trend data, or otherwise be used in creating a baseline against which to compare new user data as it is generated.

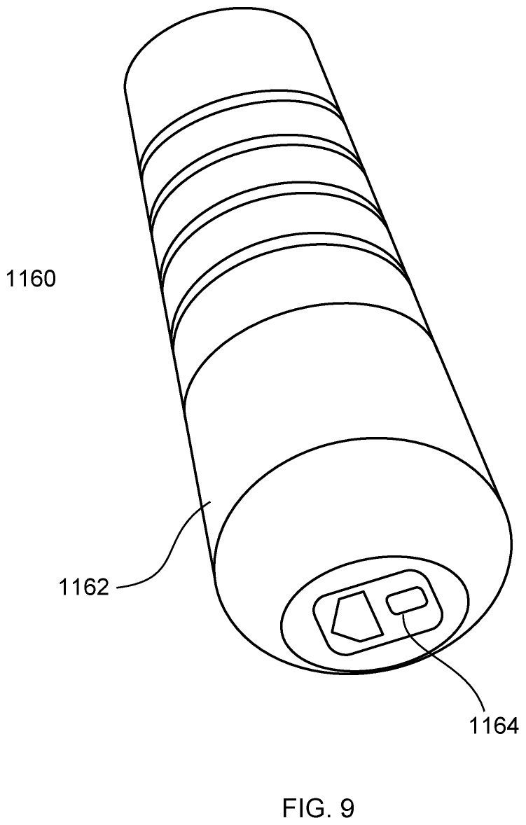

FIG. 9 shows an embodiment of a handle that could accompany a toilet. Handle 1160 includes electrical lead 1162 and PPG sensor 1164. Electrical lead 1162 could be a lead for a bioimpedance sensor and/or an ECG sensor. In one preferred embodiment, a handle would be connected to a cord (with wiring) that connects to the toilet. Alternatively, a handle could be wireless and in digital communication with a controller that is also in digital communication with other of the toilet sensors. And another preferred embodiment a handle would be mounted to a structure adjacent to the toilet bowl. In either embodiment a second handle they also do used. A second handle may originate from the same connection point to the toilet or a location symmetrically opposite or mirrored from the first handle.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. A toilet comprising:
a bowl having a volume of water for receiving excreta from a user, the excreta and water forming a volume of wastewater;
a first sensor for detecting the addition of excreta to the water in the bowl;
an orifice for collecting a sample of wastewater;
wherein the sample is analyzed to determine a property of the excreta, which is used to obtain health and wellness information about the user.

2. The toilet of claim 1, wherein the orifice is below the level of the volume of water.

3. The toilet of claim 1, wherein the wastewater is filtered based on a size threshold that corresponds to a target biomarker.

4. The toilet of claim 1, wherein the wastewater is filtered based on ionic charge.

5. The toilet of claim 1, wherein the analysis comprises a test to detect a pathogen.

6. The toilet of claim 1, wherein the analysis comprises a test to detect a biomarker.

7. A method of collecting and testing an excreta sample from a user in a toilet comprising:
providing a toilet comprising:
a bowl having a volume of water for receiving excreta from a user, the excreta and water forming a volume of wastewater;
a first sensor for detecting the addition of excreta to the water in the bowl;
an orifice for collecting a sample of wastewater;
receiving excreta into the bowl;
creation of wastewater through water contacting the excreta and excreta matter dispersing into the water;
drawing a portion of the wastewater into the orifice to collect a sample of the wastewater; and
testing the sample to gather health and wellness information about the user derived from the excreta matter in the wastewater.

8. The method of claim 7, wherein the orifice is below the level of the volume of water.

9. The method of claim 7, wherein the sample is collected before the toilet acts to move the other wastewater from its initial location.

10. The method of claim 7, wherein the orifice is in the bowl.

11. The method of claim 7, further comprising an additional space in fluid connection with the bowl which also contains the wastewater and wherein the orifice is in the additional space.

12. The method of claim 7, wherein the testing comprises a test for a pathogen.

13. The method of claim 12, wherein the testing comprises a test for a bacteria.

14. The method of claim 12, wherein the testing comprises a test for a virus.

15. The method of claim 14, wherein the virus is a coronavirus.

16. The method of claim 7, wherein the remaining wastewater is moved from the bowl to a secondary processing area within the toilet.

17. The method of claim 7, wherein the sample is filtered based on the size of a target biomarker or analyte.

18. The method of claim 7, wherein the toilet further comprises a sensor for testing the sample.

19. The method of claim 18, wherein the sensor comprises a spectrometer.

20. The method of claim 18, wherein the sensor comprises a nano-FET.
